(19) United States
(12) Patent Application Publication
(10) Pub. No.: US 2021/0386551 A1
(43) Pub. Date: Dec. 16, 2021

(54) PROSTHETIC KNEE SPACER AND METHOD OF USING THE SAME

(71) Applicant: ForCast Orthopedics, Inc., Longmont, CO (US)

(72) Inventors: Jeffrey Paul CASTLEBERRY, Longmont, CO (US); Jared Ruben Hillel FORAN, Denver, CO (US); Keir HART, Lafayette, CO (US)

(73) Assignee: ForCast Orthopedics, Inc., Longmont, CO (US)

(21) Appl. No.: 17/284,868

(22) PCT Filed: Nov. 1, 2019
(86) PCT No.: PCT/US2019/059365

§ 371 Date: Apr. 13, 2021

Related U.S. Application Data
(60) Provisional application No. 62/754,049, filed on Nov. 1, 2018.

(51) Int. Cl. A61F 2/38; A61F 2/48

(52) U.S. Cl. CPC A61F 2/389; A61F 2/3859; A61F 2002/3068; A61F 2002/3863; A61F 2/482

(57) ABSTRACT

A spacer for a knee replacement prosthesis. The spacer may include a lower surface, which has a locking component adapted to interlock with a tibial tray, an upper surface, the upper surface having a pair of condyle support platforms; and a frame positioned therebetween for anchoring the upper and lower surfaces thereto, the frame having a hollowed center defined by internal cavities. In some exemplary embodiments, the spacer may further include a fluid delivery system for localized antibiotic treatment to the surrounding joint. The delivery system may include a reservoir disposed within the internal cavity, a septum operatively connected to the reservoir, and fluid delivery element for dispensing a liquid medicament from the reservoir to the surrounding infection. The fluid delivery system may interact with external components to refill, communicate with, and control the system.

### BACKGROUND

Though infection is an uncommon complication of arthroplasty, it may have devastating consequences, both physical and economic, for a patient and for the healthcare system. Infection following total knee arthroplasty can be difficult to diagnose, and is often difficult to treat once it has been diagnosed. The revision procedure that must be undertaken once an infection has been identified typically involves a combination of surgical debridement to decrease the bacterial bioload, revision of one or more components of the prosthesis, and prolonged intravenous (IV) and/or oral antibiotics to eliminate the remaining bacteria. This will mean, for the patient, a longer operating time, greater blood loss, and more chance for other complications to arise, along with increases in the total number of hospitalizations, duration of hospitalization, total number of operations, total hospital costs, and the total outpatient visits and expenses.

Currently, several options exist for the treatment of an infected total knee arthroplasty. The first option is simple suppression of the infection with IV and/or oral antibiotics. This option is generally reserved for patients that are thought, for any number of reasons, to be unfit for surgery. Simple IV and/or oral antibiotic treatment of an infected total knee arthroplasty without concomitant surgery is unlikely to result in eradication of an infection, but may suppress the infection such that it is minimally symptomatic.

A second option is an irrigation and debridement and polyethylene exchange (IDPE). In this procedure, an open irrigation and debridement of the infected knee is undertaken, with concomitant removal of the polyethylene spacer and placement of a new polyethylene spacer (a “polyethylene exchange”). In some instances, surgeons may elect to add dissolvable antibiotic beads to the knee at the time of surgery. Following this procedure, patients are generally placed on at least 6 weeks of IV antibiotics and may then be put on oral antibiotics for an indefinite period of time. The major advantage of this procedure is that it preserves the current metallic prosthesis, thus minimizing the morbidity of removing a well-fixed prosthesis. Removing a well-fixed prosthesis generally results in loss of variable amounts of native bone stock about the femur and tibia, which is of obvious detriment to the patient. The major disadvantage is that it may be difficult to eradicate the infection using this technique. The success rate for eradication of infection varies a great deal, from 31% to 75%. See, e.g., S. M. Odum, T. K. Fehring, & A. V. Lombardi, et al., “Irrigation and debridement for periprosthetic infections: does the organism matter?” 26 J. Arthroplasty 114-118 (2011); I. Byren, P. Bejon, & B. L. Atkins, et al., “One hundred and twelve infected arthroplasties treated with ‘DAIR’ (debridement, antibiotics and implant retention): antibiotic duration and outcome,” 63 J. Antimicrob. Chemother. 1264-1271 (2009).

The third option is a so-called “two-stage exchange.” A two-stage exchange consists of two operations. In the first operation, the existing prosthesis and surrounding cement are both removed, a thorough irrigation and debridement is performed, and an antibiotic-eluting polymethylmethacrylate (PMMA) (“bone cement”) temporary spacer is placed in place of the prosthesis. Multiple options for a replacement temporary spacer may exist for this procedure. For example, the temporary spacer may be a static spacer, which consists of a block of PMMA that spans the tibiofemoral space and as such holds the knee in a fixed extended position. The temporary spacer may also be of the articulating variety; in this case, the femoral, tibial, and polyethylene parts of the knee are replaced with antibiotic-impregnated molded PMMA components, which may function as a temporary prosthesis, and which may temporarily elute a high, but ever diminishing concentration of antibiotics into the knee. This articulating device allows for some movement of the knee joint. There are several commercially available varieties of PMMA articulating spacers, some of which come pre-formed and pre-loaded with antibiotics (for example, Interspace Knee, Exactech, Gainesville, Fla.) and some of which are molded by the surgeon in the operating room (for example, Stage One, Zimmer Biomet, Warsaw, Ind.). Additionally, each of these devices aim to temporarily replace the infected prosthesis. That is, the metal femoral and tibial components are removed and replaced with a temporary femoral and tibial drug delivery implant. Following the first stage, in which the existing prosthesis is replaced with a temporary prosthesis, the patient is placed on at least 6 weeks of IV antibiotics. When the infection is thought to be eradicated, the second stage of the procedure is performed. In this stage, the PMMA spacer is removed, and replaced with a revision prosthesis. The advantage of a two-stage procedure is that it has a relatively higher success rate, ranging from 72% to 93%, but is still perceived and deficient in treating these patients. See, e.g., S. M. Mortazavi, D. Vegari, A. Ho, B. Zmistowski, & J. Parvizi, “Two-stage exchange arthroplasty for infected total knee arthroplasty: predictors of failure,” 469 Clin. Orthop. Relat. Res. 11:3049-54 (November 2011); F. S. Haddad, M. Sukeik, & S. Alazzawi, “Is single-stage revision according to a strict protocol effective in treatment of chronic knee arthroplasty infections?” 473 Clin. Orthop. Relat. Res. 1:8-14 (January 2015). The disadvantages are the morbidity of two major operations, potential bone loss caused by removal and reimplanation of the prosthesis, a difficult period for the patient when the antibiotic spacer having restricted functionality is in place, and the high cost of revision implants.

A fourth option is a so-called “one-stage” or “single-stage” exchange. In one-stage exchange arthroplasty, the infected metal prosthesis is removed, the joint is thoroughly irrigated and debrided, and a new revision prosthesis is put in place (often with antibiotic cement for fixation) all in one operation. This is uncommon in the United States for fear of failure given placing a new, sterile prosthetic in an infected wound bed. If this approach is undertaken, generally a large amount of tissue and bone are resected, which is a clear disadvantage.

In addition, there are numerous other therapeutic procedures advanced that rely on delivery of medicaments directly into a major joint capsule, e.g. the synovial fluid within joint. Steroids or analgesics are commonly injected into the joint space to treat pain. Research therapies using hyaluronic acid or stem cells injected directly into the joint space of artificial joints are being evaluated to reduce detrimental conditions and improve patient outcomes. In each of these alternative therapies, the ability to locally deliver the medicament to achieve a minimum therapeutic dose over time is a significant advantage over current medicament delivery methods.

### SUMMARY

According to an exemplary embodiment, a spacer for a knee replacement prosthesis may be provided herein. The spacer may form an orthopedic prosthesis designed for surgical implantation into a knee joint between a femoral condyle and corresponding tibial plateau. The spacer may include a lower surface, the lower surface having a locking component adapted to interlock with a tibial tray; an upper surface, the upper surface having a pair of condyle support platforms with respective articular geometries; and a frame positioned therebetween for anchoring the upper and lower surfaces thereto, the frame having a hollow outer portion surrounding an internal cavity. In some exemplary embodiments, the spacer may further include a fluid delivery system for localized drug treatment to the surrounding joint. In some exemplary embodiments, the spacer may further include a sensor and a communication system for monitoring joint health in the patient and communicating that information to an external monitor, e.g. a smart phone with a Bluetooth wireless connection. The fluid delivery system may include a reservoir disposed within the internal cavity, a septum operatively connected to the reservoir, and fluid delivery means for dispensing a liquid medicament, such as an antibiotic, from the reservoir to the surrounding infection. The fluid delivery system may further interact with components and systems external to the knee or skin to refill and control said system.

According to another exemplary embodiment, a total knee prosthesis, used to perform a total knee arthroplasty, may be provided herein. The total knee prothesis may include an artificial femoral component having a pair of condylar surfaces secured to the distal end of a femur; an artificial tibial component having a tibial tray secured to the proximal end of a tibia; and a prosthetic knee spacer, the spacer having a lower surface, the lower surface having a locking component adapted to interlock with the tibial tray; an upper surface, the upper surface having a pair of condyle support platforms, each condyle support platform adapted to support one of the condylar surfaces; and a frame positioned therebetween for anchoring the upper and lower surfaces thereto, the frame having a hollow outer portion surrounding an internal cavity. In some exemplary embodiments, the spacer may further include a fluid delivery system for localized drug treatment to the surrounding joint. In some exemplary embodiments, the spacer may further include a sensor and a communication system for monitoring joint health in the patient and communicating that information to an external monitor, e.g. a smart phone with a Bluetooth wireless connection. The fluid delivery system may include a reservoir disposed within the internal cavity, a septum operatively connected to the reservoir, and fluid delivery means for dispensing a liquid medicament, such as an antibiotic, from the reservoir to the surrounding infection. The fluid delivery system may further interact with components external to the knee or skin to refill and control said system.

According to another exemplary embodiment, a method for adding a liquid medicament to a prosthetic knee spacer may be provided. The spacer may include a lower surface, the lower surface having a locking component adapted to interlock with a tibial tray; an upper surface, the upper surface having a pair of condyle support platforms with respective articular geometries; and a frame positioned therebetween for anchoring the upper and lower surfaces thereto, the frame having a hollow outer portion surrounding an internal cavity. In an exemplary embodiment, the frame may provide stiffness, load strength and fatigue strength, the upper surfaces may provide a lubricous contact against the metal femoral condyles to enable wear resistance in use and prolong life of the implant, and the lower surface may enable interlocking with the tibial tray through either plastic deformation or through elastic snaps. In some exemplary embodiments, the spacer may further include a fluid delivery system for localized drug treatment to the surrounding joint. The fluid delivery system may include a reservoir disposed within the internal cavity, a septum operatively connected to the reservoir, and fluid delivery means for dispensing a liquid medicament, such as an antibiotic, from the reservoir to the surrounding synovial fluid, soft tissues, cartilage, bone and surfaces of the total joint implant that may be supporting the infection. The fluid delivery system may further interact with components and systems external to the knee or skin to refill and control said system. The method of adding fluid to said spacer may include applying an external force to the septum; inserting a hollow tube into the septum; and inserting fluid into the septum via the hollow tube. Such a method may be performed percutaneously or otherwise, for example to initially fill the spacer.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is an exploded view of an exemplary embodiment of a prosthetic knee spacer;

FIG. 2 is a graphical representation of comparative antibiotic concentrations in synovial fluid by different drug delivery mechanisms, including a typical eluting cement spacer, daily catheter injections, and an exemplary embodiment of a prosthetic knee spacer in accordance with the present invention;

FIG. 3 is an exemplary embodiment of an intraarticular injection into a prosthetic knee spacer;

FIG. 4 is an exemplary embodiment of a filling system for monitoring injection pressure;

FIG. 5 is an exemplary embodiment of a control system for use in conjunction with a prosthetic knee spacer;

FIG. 6 illustrates an exemplary embodiment of a reservoir;

FIG. 7 is an exemplary embodiment of a septum;

FIG. 8 is an exemplary embodiment of a passive diffuser; and

FIG. 9A illustrates an exemplary embodiments of an active pumping mechanism.

FIG. 9B illustrates an exemplary embodiments of an active pumping mechanism.

FIG. 9C illustrates an exemplary embodiments of an active pumping mechanism.

FIG. 9D illustrates an exemplary embodiments of an active pumping mechanism.

FIG. 9E illustrates an exemplary embodiments of an active pumping mechanism.

### DETAILED DESCRIPTION OF THE EMBODIMENTS

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention,” “embodiments,” or “invention” do not require that all embodiments of the invention include the discussed feature, advantage, or mode of operation.

According to at least one exemplary embodiment, and referring generally to the Figures, a spacer for a knee replacement prosthesis may be shown and described herein. The spacer may form an orthopedic prosthesis designed for surgical implantation into a knee joint between a femoral condyle and corresponding tibial plateau. The spacer may include a drug delivery system capable of dispensing liquid medicaments, in a controlled and titratable manner, directly into a synovial joint and adjacent tissue(s), thereby providing localized treatment to an infected joint after an infected total knee arthroplasty revision procedure, and in some cases after a primary arthroplasty procedure for post-operative prophylactic antibiotic treatment. The spacer may rely on a liquid medicament supply that can be replenished during treatment without the need for post-treatment surgery to remove and/or replace the spacer device. The spacer may also be shaped and configured so as not to interfere with the function of the knee joint of the patient. Such a spacer may create a new option for the treatment of periprosthetic knee infection, combining many of the benefits of the treatments of the previous options and eliminating many of the drawbacks inherent in said treatments.

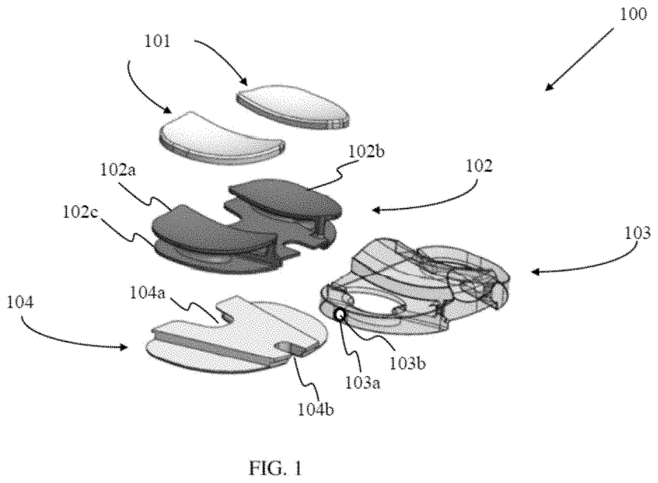

FIG. 1 may depict an exploded view of an exemplary embodiment of a prosthetic knee spacer 100 in accordance with the present invention. The prosthetic knee spacer 100 may be designed for surgical implantation into a knee joint between a femoral condyle and corresponding tibial plateau. In some exemplary embodiments, the prosthetic knee spacer 100 may be utilized as part of a total knee arthroplasty implant, configured to fit between a femoral component and a tibial component of a total knee replacement prosthesis. In combination with conventional total knee arthroplasty implants, the knee spacer 100 may be designed to comply with industry standards, including but not limited to, ASTM F2777-16, ASTM F1800-12, and ISO 14243-3, and as further described herein.

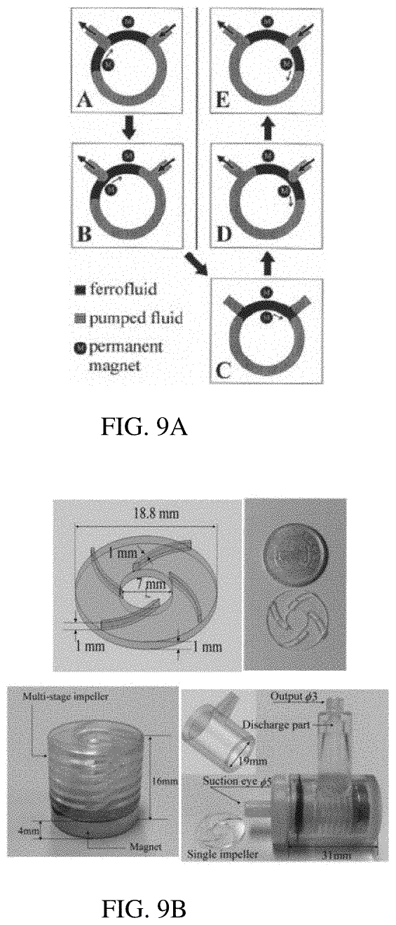

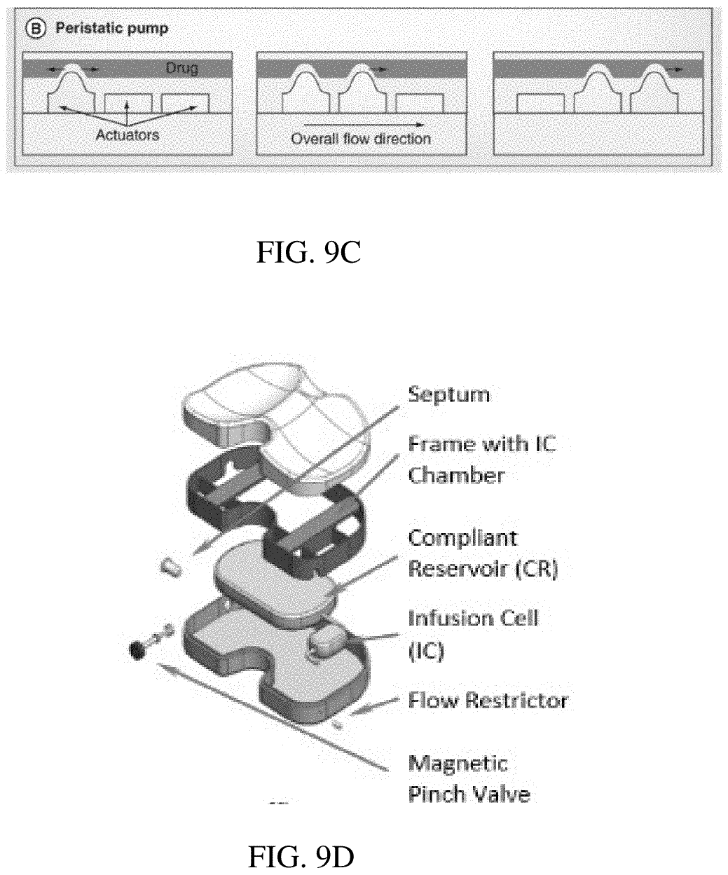

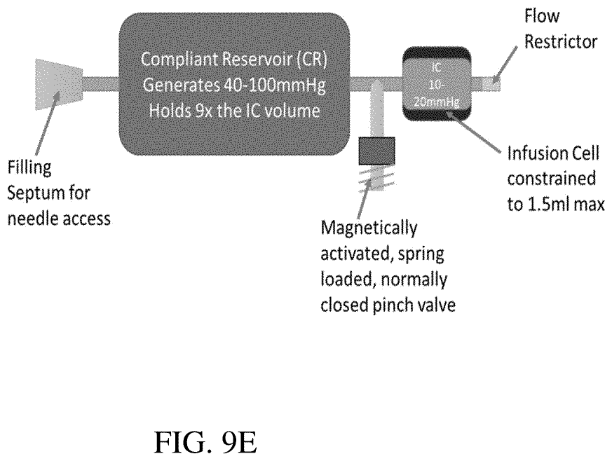

In some exemplary embodiments, the prosthetic knee spacer 100 may include a lower surface 104, the lower surface having a locking component 104a and 104b adapted to interlock with a tibial tray; an upper surface 101, the upper surface having a pair of condyle support platforms with respective articular geometries; and a frame 102 positioned therebetween for anchoring the upper and lower surfaces 101, 104 thereto, the frame 102 having a generally disc-shaped center lower portion 102c configured to receive the lower surface 104 support portions on the left 102a and right 102b, each of which is configured to receive one of the pair of condyle support platforms of the upper surface 101. The frame 102 as shown if FIG. 1 is an internal frame in which internal and external, or peripheral, cavities or voids are defined between the lower portion 102c and support portions 102a, 102b and between each of the support portions. In some exemplary embodiments, frame 102 is an external frame (FIG. 9E). In some exemplary embodiments, the spacer 100 may further include a fluid delivery system for localized drug treatment to the surrounding joint. The fluid delivery system may include a reservoir 103 disposed within the internal cavity, a septum 103b operatively connected to the reservoir 103 at port 103a, and fluid delivery means for dispensing a liquid medicament, such as an antibiotic, from the reservoir 103 to the surrounding infection. The fluid delivery system may further interact with components and systems external to the knee or skin to refill, communicate with, and control said system.

As shown in exemplary FIG. 1, the frame 102 may form a generally kidney-shaped structure that corresponds to a cross-sectional shape of the proximal portion of the natural tibial bone. In particular, the perimeter may resemble a footprint with wider anterior and narrow posterior. It should be appreciated, by those of ordinary skill in the art, that the frame 102 may be formed in any suitable outer shape and/or size for simulating the tibial plateau. Such formation may account for the variation in patient knee size over the population, or configurations associated with special conditions, such as partial knee replacement or significant bone loss. In some exemplary embodiments, the spacer 100 is constructed in each of a variety of shapes and sizes; for example, the frame 102 may be made in the equivalent shapes and sizes of other commercially-available total knee prosthesis spacers, or the shapes and sizes of the total knee prosthesis spacers sold by a particular company. This may allow the exemplary prosthetic knee spacer 100 to replace existing spacers for DAIR (debridement, antibiotics, and implant retention) procedures that incorporate spacer exchange.

In some exemplary embodiments, the frame 102 may be configured to correct limb alignment, e.g., connect and extend across the spacer planar view, and distribute the condyle load to the tibial plate or tibia. Preferably, compressive forces are distributed over as large a load- bearing surface as possible, such loads being dispersed to the load-supporting perimeter atop strong cortical bone.

According to an exemplary embodiment, the frame 102 may be constructed from one or more high strength materials to minimize the mass and/or volume necessary to achieve the load and fatigue requirements typical of a major joint spacer. The frame material may thus allow for the construction of a supporting structure, wherein the center portion and periphery define hollowed-out cavities. Specifically, the frame material may provide the strength and rigidity necessary to support the primary loads and subsequent stresses occurring between the condyle contact surfaces and the tibial tray.

For example, the frame 102 may be composed of a metallic material, such as stainless steel, titanium, or cobalt chromium, formed into shape by any known methodology, such as by machining, stamping, metal-injection-molding, and the like. In an exemplary embodiment, the frame 102 is constructed from titanium in order to utilize commercially-available additive manufacturing techniques (e.g., 3D Printing) and post-processing via hot isostatic pressure. Such processing may reduce production imperfections and deliver a frame 102 with density consistency and material properties suitable for load strength, impact strength, and fatigue testing common with total knee arthroplasty implant testing to achieve regulatory clearance.

In some exemplary embodiments, the prosthetic knee spacer 100 may be customized to fit a specific patient's knee joint. In particular, the spacer 100 may be designed (using CAD and FEA to iterate the size, shape and location of the frame struts and features in order to achieve acceptable predicted stresses) to withstand key conditions, including the direct loads from patient weight and impact from movement, and fatigue loads from cyclic behavior over the life expectancy of the implant, as recognized by industrial standards and references. See, e.g., Bergmann et al., “Standardized Loads Acting in Knee Implants” 9 PLoS One (2014); U.S. Dept. of Health and Human Services, Food and Drug Administration, “Class II Special Controls Guidance Document: Knee Joint Patellofemorotibial and Femorotibial Metal/Polymer Porous-Coated Uncemented Prostheses; Guidance for Industry and FDA,” (2003); ASTM F1800, “Standard Practice for Cyclic Fatigue Testing of Metal Tibial Tray Components of Total Knee Joint Replacements” ASTM international, West Conshohocken, Pa. (2012); ASTM 2777, “Standard Test Method for Evaluating Knee Bearing (Tibial Insert) Endurance and Deformation Under High Flexion,” ASTM International, West Conshohocken, Pa. (2016).

In some exemplary embodiments, the frame 102 may be designed to remove as much of the spatial volume (i.e., generate as much material void) as possible, in the frame 102 in order to provide space for the fluid delivery system. This step may be facilitated by both Computer Aided Design (CAD) modeling and Finite Element Analysis (FEA), wherein the design can be molded with specific dimensions and material properties. Boundary conditions for simulating loading of the standards can be exerted in the model and the resulting FEA can indicate the locations of high stress and their magnitude which can be compared to published material properties, including considerations of safety factor, etc.

Stress risers may be identified (typically corners, thin walls, etc.), and can be modified in CAD by adding radii, chamfers or adjusting dimensions, in order to reduce the predicted stresses to a level believed to be sufficiently lower than the published material property limits. Samples may be manufactured, treated, and subjected to testing on appropriate equipment per the standards. The results from the testing can then be compared to the FEA results and adjustments to the FEA/CAD modeling may be performed to improve correlation. Subsequent design refinements can be adjusted within the CAD model with greater confidence of good testing outcomes. Further refinements may be pursued, as needed, to ensure that the related standard requirements have been met with suitable safety factor/margin, and that the material void of the frame 102 is maximized to facilitate both placement and assembly of the fluid delivery system.

According to an exemplary embodiment, the upper surface 101 of the prosthetic knee spacer 100 may connect to the top of the frame 102 via frame struts and supports extending upwardly therefrom. The upper surface may mechanically attach (e.g., with screws), lock, or adhere to the frame 102 to avoid micromotion between the frame 102 and the upper surface 101 so that debris is not generated in situ. The prosthetic knee spacer 100 may utilize any suitable mechanical lock known in the industry, including but not limited to, snaps, dovetail joints, and the like. In some exemplary embodiments, the spacer 100 may further incorporate biocompatible adhesives or cements, such as bone cement, if required to achieve the needed attachment performance, as would be understood by one of ordinary skill in the art.

The upper surface 101 may accommodate a femoral component of a knee replacement prosthesis. In some exemplary embodiments, the upper surface 101 may include a pair of smooth-surfaced and slightly concave condyle support platforms disposed on opposite sides of the frame 102 at support portions on the left 102a and right 102b . Each of the condyle support platforms may be used to, for example, accommodate the condyles of the femoral component, such that each of the condyles of the femoral component fits within one of the concave platforms. In some exemplary embodiments, the curvature of the concave platforms may be substantially molded to match existing metal femoral components in existing knee implants.

According to an exemplary embodiment, the upper surface 101 may be fabricated from the identical, modified Ultra High Molecular Weight Polyethylene (UHMW-PE) materials used in current solid spacers. The UHWM-PE materials can be crosslinked with or without additives, such as Vitamin E, to reduce oxidation and increase wear life. The upper surface 101 may provide a lubricious surface in contact with the metal femoral condyles, and wear life suitable to support the life expectancy of the implant. See, e.g., ISO 14243, “Implants for surgery—Wear of total knee-joint prostheses,” International Organization for Standardization (2009).

The height of the prosthetic knee spacer 100 may be adjustable via modification of the UHMW-PE material thickness of the upper surface 101. For example, an operating surgeon may attach the upper surface 101 within the sterile field to fit the modular knee spacer 100 to patient needs, allowing the use of a common frame 102 to generate a 12 mm, 14 mm, 16 mm, and 18 mm spacer height with different surface components (e.g., thickness) with the common frame 102.

According to an exemplary embodiment, the prosthetic knee spacer 100 may further include a lower surface 104, the lower surface having a locking component 104c adapted to interlock with a tibial tray. The lower surface 104 may be fabricated from the same, modified UHMW-PE materials used in current solid spacers. The lower surface 104 may mechanically lock to the frame so that debris is not generated in situ. The lower surface 104 may mimic the tibial tray lock of solid PE spacers to reduce any risk of relative micromotion between the spacer 100 and the tibial tray. Micromotion between these parts can generate wear and particulate that can cause the joint to fail overtime. Duplicating this locking design (PE surface in contact with the metal tibial tray) and function minimizes that risk. For example, the prosthetic knee spacer 100 may utilize any suitable mechanical lock known in the industry, including but not limited to, snaps, dovetail joints, and the like. In some exemplary embodiments, the spacer 100 may further incorporate biocompatible adhesives or cements, such as bone cement, if required to achieve the needed performance, as would be understood by one of ordinary skill in the art.

For the permanent configuration, the prosthetic knee spacer 100 may also incorporate mechanical components that allow a surgeon to properly install and remove the prosthetic knee spacer 100 from the corresponding tibial tray. In many instances, the surgeon may use a tool and hammer, impacting the spacer 100 to deform it slightly and engage the locking mechanism. Alternatively, the manufacturer may provide a specialized hand tool to facilitate placement of the spacer 100 and engagement of the lock. The prosthetic knee spacer 100 may incorporate UHMW-PE protective surfaces and conventional PE interfaces to facilitate use with existing implants.

For a temporary configuration, the lower surface 104 may be fabricated to allow secure bonding directly to the tibial plateau with bone cement, whereby the spacer 100 acts as a temporary treatment device when a tibial plate is not available.

It should be noted that the prosthetic knee spacer 100 can be implemented without a fluid delivery system. Some of the potential benefits of the system, in the absence of a fluid delivery system, may include: (1) reduced cost given the complexity of chemically modifying the PE, crosslinking, machining and/or molding; (2) better wear-life and/or device performance (e.g., an altered configuration, such as a thinner section of modified UHMW-PE, may prove better at preventing oxidation, delamination and/or cold-flow of the material); (3) the ability to provide other clinically beneficial assemblies or components within the void-space, including but not limited to, a wireless communication monitoring system with sensors for detecting device failure, joint infection, and/or other beneficial early warning indications; and (4) an adjustment system for modifying the height and position of the spacer in situ without surgery.

An advantage of the present invention is that the prosthetic knee spacer 100 can be implanted and maintained over the life of the patient or entire implant (whichever is shorter) without necessitating its removal and/or replacement. Such advantage remains true regardless of the substance placed within the voided frame. The present invention can thus eliminate the need for revision knee surgery common with antibiotic impregnated cement spacers, and the like. Such advantage also reduces patient morbidity and significantly reduces healthcare costs associated with these patient treatments.

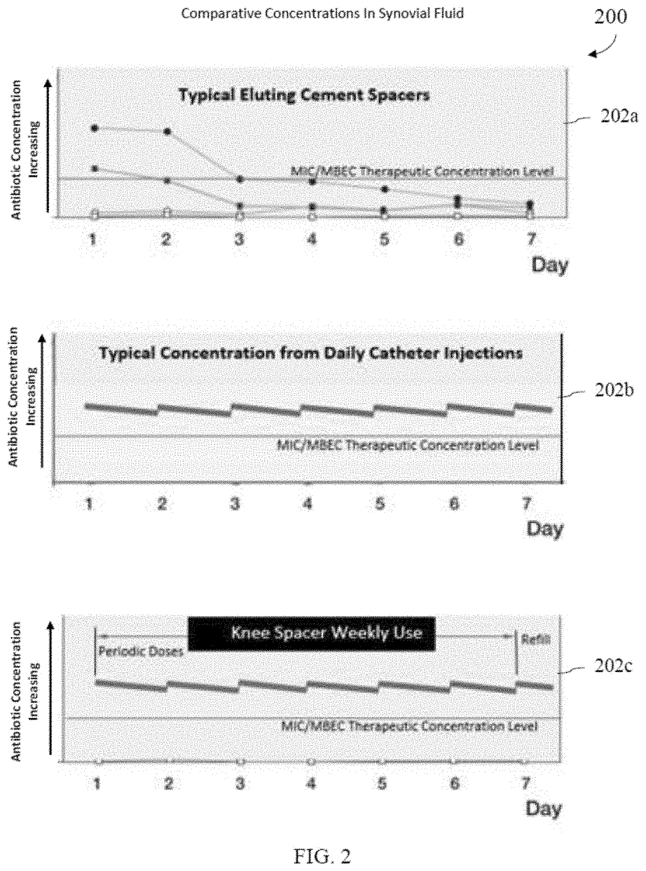

FIG. 2 may illustrate a comparative graph 200 of antibiotic concentrations in synovial fluid by different drug delivery mechanisms, including a typical eluting cement spacer, daily catheter injections, and an exemplary embodiment of a prosthetic knee spacer in accordance with the present invention.

Prosthetic joint infection (PJI), or periprosthetic infection, remains a serious complication in orthopedic surgery, with approximately 1-2% incidence in total knee arthroplasty (TKA). PJI management commonly involves a prolonged course of antibiotics, often with surgical intervention, such as a debridement, antibiotic, irrigation, and implant retention (DAIR) procedure with spacer exchange; one or two-stage revision arthroplasty; arthrodesis; and amputation. Such bacterial infection typically requires continuous antibiotic therapy for a minimum of 6 weeks to effectively eradicate the infection. Moreover, therapeutic concentrations of antibiotics must be maintained throughout treatment in order to address the bacteria lifecycle replication. When antibiotic concentrations fall to sub-therapeutic levels, surviving bacteria can recolonization, which can serve as a nidus for biofilm formation.

Consequently, therapeutic decision-making must not only consider minimum inhibitory concentrations (MIC) of antibiotics but also the minimum biofilm eradication concentration (MBEC). As described herein, the term “minimum inhibitory concentration” (MIC) refers to the lowest concentration of an antimicrobial that will inhibit the visible growth of a microorganism after overnight incubation. The term “minimum biofilm eradication concentration” (MBEC) refers to the lowest concentration of an antimicrobial that eradicates 99.9% of the colony-forming units (CFU), (i.e. a three-log reduction) in a bacterial biofilm, compared with growth controls in the same conditions.

As shown in exemplary FIG. 2, each of the graphs 202a, 202b, 202c represents antibiotic concentrations in synovial fluid after drug administration by one of three different drug delivery mechanisms. In particular, graphs 202a, 202b, and 202c demonstrate concentrations provided from an antibiotic-loaded cement spacer, a regimen of daily catheter injections, and exemplary prosthetic knee spacer, respectively.

One of the primary advantages of the present invention over competitive therapies (e.g., fixed eluting implants) is the ability to maintain therapeutic antibiotic concentrations in synovial fluid over an elongated period. As illustrated in graph 200c, the prosthetic knee spacer may offer a localized drug delivery system that can achieve high, therapeutic concentrations of antibiotic in synovial fluid. Such antibiotic concentrations may be maintained over an elongated therapy period, typically between a 6-week to 6-month period, or as desired, due to its ability to be refilled with antibiotic. This may be an advantage over some conventionally employed systems, such as short-life antibiotic impregnated cement spacers or time-release dissolving beads, that cannot maintain therapeutic antibiotic concentrations over a sufficient duration for complete treatment. The benefit of achieving high concentrations can be demonstrated by published clinical studies of daily intraarticular catheter injection by Dr Leo Whiteside, whose patient outcomes were considerably better for eradicating periprosthetic joint infection (PJI) than traditional 2-Stage Revisions using impregnated cement spacers.

In some exemplary embodiments, the knee spacer may deliver high concentrations of medicament via micro-bolus delivery from a pumping system. In other exemplary embodiments, the prosthetic knee spacer may achieve nominal therapeutic concentrations through diffusion.

Another advantage of the present invention is the reduced patient/provider burden of repeated painful injections into the knee space. Although daily injections may maintain therapeutic concentrations (as shown in graph 202b), the burden of repeated injections may result in inflammation and pain. The present invention solves this problem by having an onboard fluid delivery system and larger-volume reservoir. The reservoir need not be refilled at the same interval, as daily injections, reducing the burden on the patient and provider.

The use of a liquid form of antibiotic that the surgeon selects and fills (and refills) into the drug delivery system of the prosthetic knee spacer allows the surgeon to match the type of antibiotic with the bacteria detected from evaluation and diagnosis of knee-tap samples. In some exemplary embodiments, the spacer can also provide post-surgical administration of antibiotics after primary total knee arthroplasty to help reduce the incidence of acute PJI in patients undergoing their first knee replacement.

In some exemplary embodiments, the fluid delivery system can be used for liquid medication and treatment other than antibiotics, such as steroids, stem cells, and hyaluronic acid.

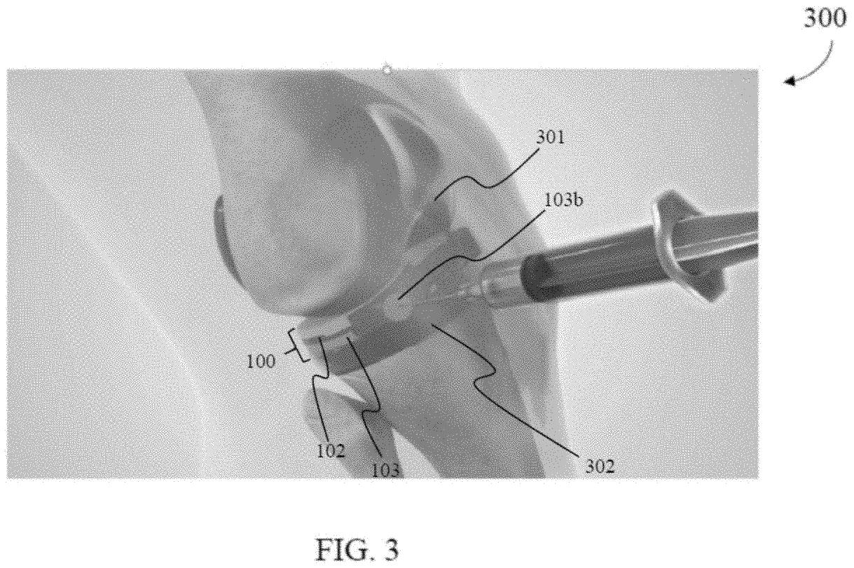

FIG. 3 may depict an exemplary embodiment of a percutaneous injection 300 into the prosthetic knee spacer 100 for refilling the internal reservoir 103. The prosthetic knee spacer 100 is shown implanted between a femoral component 301 and a tibial component 302, and in an exemplary embodiment is part a total knee replacement prosthesis. As described herein, the prosthetic knee spacer 100 may include an implantable fluid delivery system disposed within the frame 102. The fluid delivery system may be accessible from an external source, e.g., a syringe attached to a needle, via a filling septum 103b or port 103a. In some exemplary embodiments, for example, the septum 103b or port 103a is slightly proud or extended from the edge of the space to facilitate finding its location by palpation and concurrently minimizing the tissue thickness and injection depth that must be achieved to refill the fluid delivery system. In some exemplary embodiments, for example, a soft, elastomeric septum 103b may allow for needle penetration to facilitate percutaneous injection for refilling the reservoir 103 through the septum 103b. The filling septum 103b may connect to the reservoir within the fluid delivery system and prevent leakage of the concentrated liquid medicament.

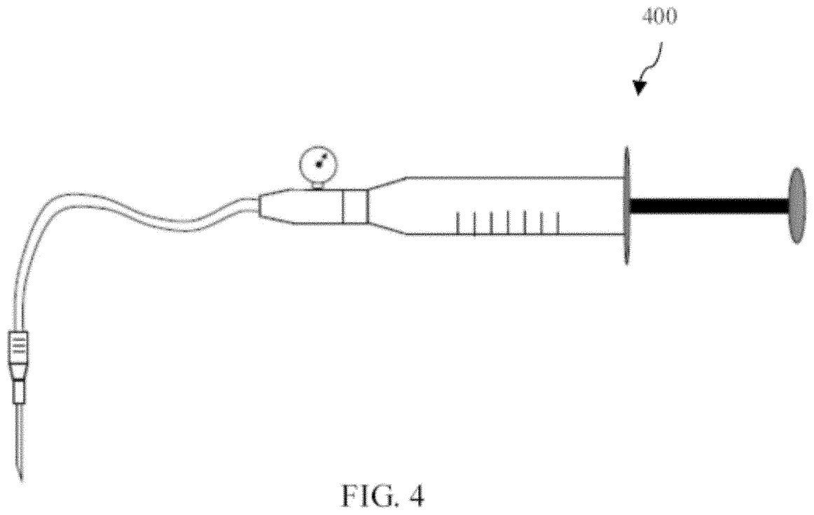

According to an exemplary embodiment, the septum 103b may be protuberant from the surface of the spacer 100, which may, for example, facilitate palpation of the septum 103b by an administrating physician. The reservoir 103 may be refilled after the spacer 100 is implanted, by the application of a percutaneous needle. An administrating physician may insert the needle through the skin of the patient and into the septum 103b of the spacer 100, thereby allowing access to an internal reservoir 103 of the spacer 100 and allowing the spacer 100 to be refilled via the needle, for example with the use of a syringe or pressure monitoring gauge 401 (see FIG. 4). According to an exemplary embodiment, the septum 103b may be constructed of an implantable-grade rubber or any other suitable flexible material, such that a needle can be inserted through the septum 103b and into an internal reservoir 103 without significantly compromising the ability of the septum 103b to seal liquid medicament in the reservoir 103. For instance, in some exemplary embodiments, the septum 103b may be capable of being punctured over 180 times without reservoir leakage.

According to another exemplary embodiment, the septum may be flexibly held in place by the spacer 100, such that the application of a force to the septum 103b, with a needle or other tool, can open the septum 103b and allow for an internal reservoir 103 of the spacer 100 to be refilled via a needle. For example, in an exemplary embodiment, the septum 103b may be constructed from a hard material and may be spring-loaded or otherwise held in place by elastic material. In another exemplary embodiment, the internal reservoir 103 may have a structure capable of retaining fluid without requiring a septum to act as a barrier; for example, according to an exemplary embodiment, the internal reservoir 103 may be filled with a sponge, such as a hydrophobic or hydrophilic sponge, and fluid may be injected directly into the sponge.

In another exemplary embodiment, the internal reservoir may form a “self-healing” bladder made from silicone that allows needled penetration and seals said puncture after penetration by a needle. According to such an embodiment, the septum 103b may be, for example, an opening within the hollowed center portion of the frame, configured to allow insertion of a needle into the internal reservoir 103.

According to an exemplary embodiment, the septum 103b may allow for both addition of and removal of antibiotic fluid, such that antibiotic fluid (or a mixture of antibiotic and synovial fluid constituents) may be drawn out of the spacer 100 to empty it prior to refill, as well as be added to the spacer 100; this may allow for antibiotic fluid levels or concentrations in the spacer 100 to be evaluated more easily. Solutions other than antibiotic fluid may also be added or removed, for example through the septum; for example, in one exemplary embodiment, anticoagulants may be added to prevent clogging of the pores, while in another exemplary embodiment, chemical surfactants may be added to help break down biofilm on the prosthesis. In a further exemplary embodiment, tissue tolerable antimicrobial solutions may be added or used including antiseptics, bactericides, and germicides.

According to an exemplary embodiment, the septum may have a localization marking that allows such to be localized on X-ray or fluoroscopy. For example, according to an exemplary embodiment, the septum 103b may be surrounded by a radio-dense ring that allows the septum 103b to be observed in an X-ray or ultrasound. According to another exemplary embodiment, such as when the septum 103b is constructed from a hard material, the septum 103b itself may be constructed from a radio-dense material.

FIG. 4 may illustrate an exemplary embodiment of a filling system 400 for monitoring injection pressure. At one or more instances within the treatment period in which the antibiotic dispensing spacer is in place, the spacer may become depleted past a desirable point. For example, the reservoir of the antibiotic dispensing spacer may become fully depleted, or sufficiently depleted that the amount of antibiotic being dispensed by the spacer is near a minimum desirable amount or less than a minimum desirable amount. At that point, the spacer may be refilled percutaneously, for example by the injection of a syringe into a septum of the spacer or the insertion of a catheter into a septum of the spacer connected to a syringe or external infusion pump. This may allow for the internal reservoir to be refilled without requiring the full surgical removal and replacement of the empty spacer with a filled antibiotic dispensing spacer, which may result in easier maintenance of antibiotic levels and greatly reduced inconvenience for the patient.

The filling system 400 may operate based upon the requirements of the drug delivery system. The filling system 400 may ascertain whether the reservoir is empty or nearing empty before filling to verify the fluid delivery system is properly working and minimize the potential for overfilling in error, which could damage the implant. The filling system 400 may include a pressure sensing means, and means for aspirating the remaining fluid in the reservoir before or concurrent with filling the reservoir with fresh, concentrated liquid medicament.

In some exemplary embodiments, the reservoir must be emptied (aspirated) before being refilled with highly concentrated medication to remove the diluted concentration and remnant synovial fluid mix in the reservoir before refilling. The exhausted reservoir can then simply be refilled. It is in this form that surgeons can measure reservoir pressure prior to attempting to refill. Such measurement may highlight a failure in the pumping mechanism, such as if the reservoir pressure is not low before attempting to refill.

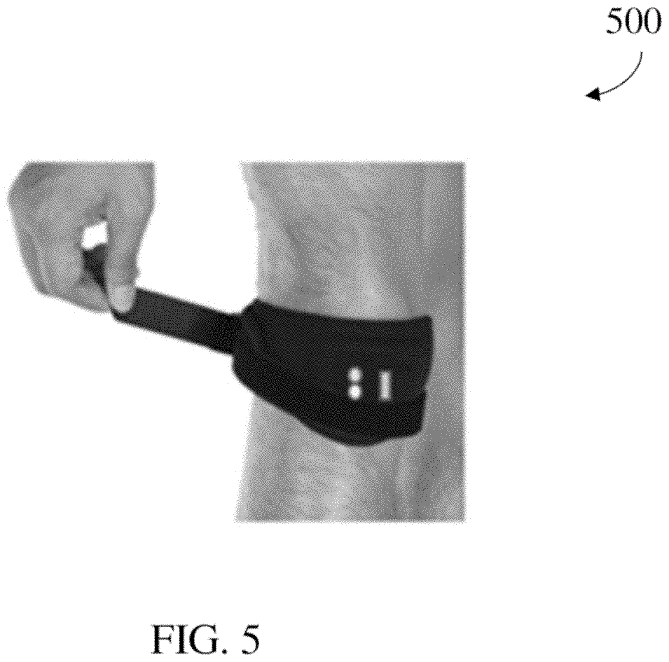

FIG. 5 may illustrate an exemplary embodiment of a control system 500 for use in conjunction with a prosthetic knee spacer. The control system 500 may be constructed as a fabric band that is configured to wrap around a patient's knee proximate the implanted spacer. The band may include a power supply, control circuitry, a locating device to ensure proper alignment, and an electromagnetic system, e.g. an electrically powered coil for the purposes of generating a localized magnetic field, or magnets, which are mounted to achieve reversable fields, i.e. rotating magnets, which interacts through the patient's skin with a magnetic component within the implanted spacer for the purpose of controlling fluid delivery from the implanted spacer. The control system 500 may facilitate a pumping mechanism, a valving mechanism, or both within the spacer. The band can be worn continuously or periodically during the treatment period, and may be discontinued after treatment is terminated (e.g., when the infection has been fully eradicated).

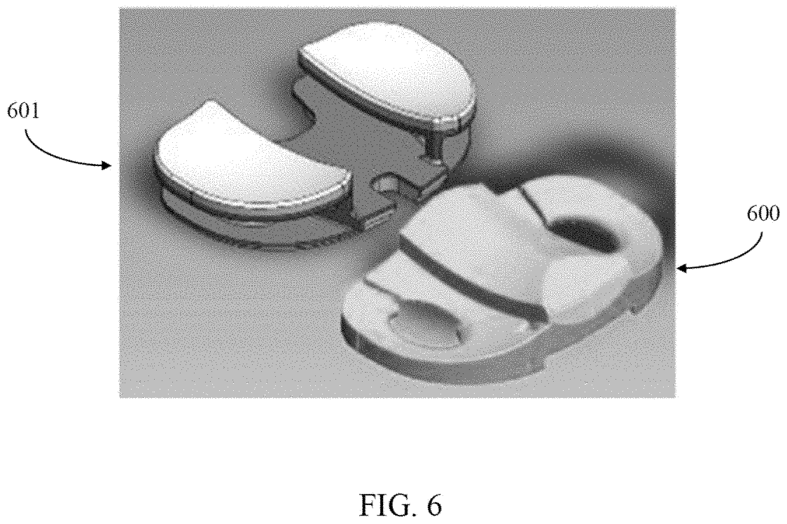

FIG. 6 may illustrate an exemplary embodiment of an internal reservoir 600 and a spacer assembly 601. The spacer assembly 601 may be a unitary structure or structure formed of a plurality of two or more components, such as internal frame 102, upper surface 101, and lower surface 104 of FIG. 1. In particular, FIG. 6 may illustrate a model of a flexible internal reservoir 600 formed as a radio frequency (RF)-welded reservoir or elastomeric bladder, illustrated in a full condition to highlight the flexible reservoir's capability to fit within the cavities of the spacer assembly 601. As illustrated in FIG. 6, this exemplary embodiment is shown to interact with a spacer assembly 601 that uses an internal frame resulting in cavities formed internally and around the periphery of the spacer assembly 601. The reservoir 600 may be formed of resin film material panels welded together along a weld line defining a seam. The reservoir 600 may define a unique shape that attempts to capture the majority of the void (i.e., internal cavities) formed within the spacer assembly 601, with or without the peripheral cavities. The fluid delivery system may reside within the welded reservoir 600 or with a smaller profile, reside external to the reservoir 600.

According to an exemplary embodiment, the reservoir 600 may hold one or more fluids, which may be, for example, solutions of antibiotics, steroids, hyaluronic acid, stem cells, anticoagulants, surfactants, other fluids, or some combination thereof, and tissue tolerable antimicrobial solutions may be added or used including antiseptics, bactericide, and germicides. The efficiency of the reservoir 600 may depend upon its ability to conform to the voided shapes of the spacer assembly 601, thereby maximizing the use of available volume for the storage of liquids. In some exemplary embodiments wherein an internal frame is employed and a peripheral cavity is formed, the flexibility of the reservoir 600 may allow its surface to expand beyond the perimeter of the spacer assembly 601 (in one or more select areas around the perimeter) in order to take advantage of additional available volume within the knee cavity that does not inhibit joint function. When therapeutic treatment ends, the reservoir 600 may collapse within, and remain surrounded by the perimeter of the spacer assembly 601, so as not to imping on the surrounding soft tissues in the knee joint. In some exemplary embodiments, as illustrated in 9E, the reservoir 600 may be segmented into various compartments to isolate different fluids until said fluids are drawn out of the reservoir 600 by the fluid delivery system.

In some exemplary embodiments, the internal reservoir 600 may form a solid, hollow container, made in conjunction with the spacer. For example, the reservoir 600 may be bonded to an internal metal frame, or similar supporting structure, to form a fluid tight reservoir that has continuous volume throughout the inner voids of the supporting structure.

In other exemplary embodiments, the internal reservoir 600 is an elastomeric bladder, wherein the components are formed by LIM, lost wax, or other molding of elastomeric materials, e.g. silicon, polyurethane, to generate a collapsed shape with no or minimal residual volume when empty. The bladder may capture the entire void space, with the fluid delivery subsystem internal to the bladder, or the bladder may be designed to fill only part of the spacer assembly 601 void space, with the fluid delivery system exterior to the bladder.

Still in other exemplary embodiments, the internal reservoir 600 may be a blow-molded or thermoformed bladder, wherein the component is molded in its expanded shape to assure minimal, if not zero, pressure generation on the reservoir contents in use. The use of a flexible or compliant reservoir 600, versus a rigid structure, is helpful in mitigating potential risk of fracture given the environmental stresses placed on the prosthetic knee spacer, especially with active patients. Either during antibiotic therapy or after therapy is complete, the reservoir 600 can sit empty/benign within the knee implant for many years, as the device will not degrade and/or fracture releasing material within the knee.

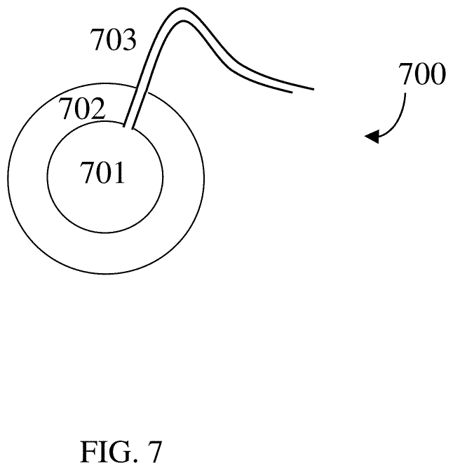

FIG. 7 may schematically illustrate an exemplary embodiment of a septum 700. As illustrated in exemplary FIG. 7, the septum 700 may include an implantable port chamber forming an opening, an internal case 701, and an external case 702 for sealing the septum 700 relative to an exterior of the device. The septum 700 may include a plurality of protrusions 703 defined about a periphery thereof that are palpable after implantation of the septum 700 in a patient to determine a relative position of the septum 700. In some exemplary embodiments, the external case may be constructed of a plastic polymer material including Polyoxymethylene (“POM”), or acetyl resin. The internal case may be constructed of titanium.

In some exemplary embodiments, the septum 700 may be a separable, implantable port connectable to the delivery device. In other exemplary embodiments, the septum 700 may be welded into or bonded onto an edge of the reservoir.

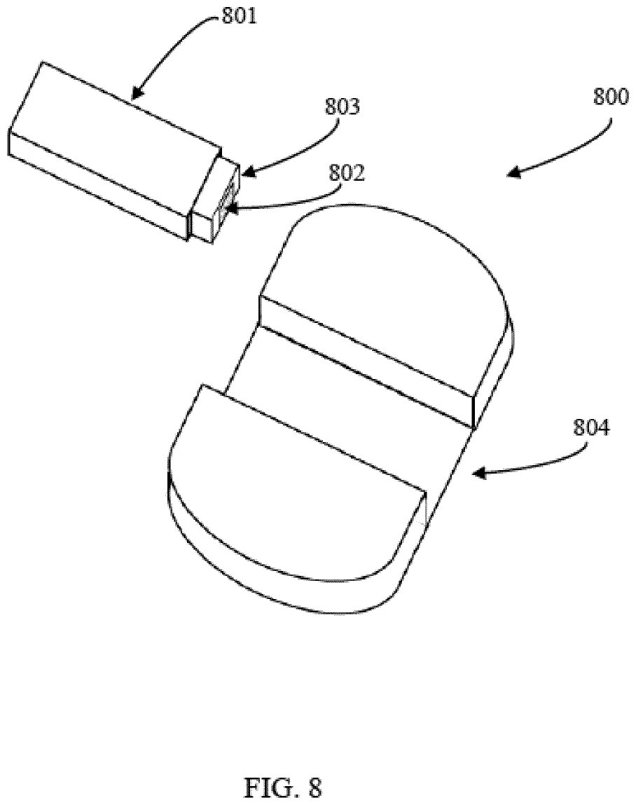

Turning now to exemplary FIGS. 8-9E, the fluid delivery system of the present invention may operate to move fluid from within the reservoir into the synovial fluid in the knee, thereby achieving therapeutic antibiotic concentrations for interaction with surrounding soft tissues, bone, cartilage, and surfaces of the total joint implant. The fluid delivery system may include means by which concentrated medicament is dosed into the patient's knee joint on a periodic basis. This can be accomplished via a passive approach, e.g. diffuser, or an active approach, e.g. pump/valve, or a combination of both. The objective is to deliver a specific volume of fluid, resulting in a specific amount of medicament as concentrated solution, in a specific timeframe to achieve and maintain a therapeutic concentration of medicament within synovial fluid and the surrounding tissues throughout therapy.

FIG. 8 may illustrate an exemplary embodiment of a spacer 800 utilizing a diffuser for passive diffusion of liquid medicament working with osmotic diffusion barriers.

The diffuser may allow simple diffusion of a liquid located within reservoir 801 of the spacer 800 to the surrounding joint, without requiring the action of a pump to dispense liquid out of the pores. The diffuser may be placed on the reservoir in one or more locations depending on the surface area needed to affect the proper dose rate. An exemplary embodiment is indicated where the diffusor 802 is placed inside a diffuser housing 803 which is attached to the reservoir 801 such that there is fluid communication between the internal reservoir and the diffusor surface. Continuing with this example, the diffusor/reservoir assembly is placed within a cavity in the remaining spacer 800, diffuser frame 804. The diffuser 802 includes a diffusion barrier that may be constructed of any suitable materials, and/or coatings, to avoid films (i.e., biofouling) being formed on the exterior surface. For example, the film may be constructed of lipids, proteins, etc., found in synovial fluid. The passive diffusion method may provide simplicity in componentry and likely repeatability/reliability if a suitable diffusion barrier can be identified/implemented. In this condition, the flexible reservoir 801 may not apply any compression on the concentrated liquid medicament therewithin, as diffusion requires fluid flow in both directions over time. With diffusion, the therapeutic concentration in synovial fluid may increase over time from a lower level to a steady state level. The internal reservoir 801 can then be refilled before concentration levels can decline below therapeutic levels.

Alternatively, the dispensing mechanism for fluids may involve active delivery flow, including one or more pumping systems housed within the implanted spacer. The internal pumping system may be powered by, for example, a power source, as desired. Exemplary embodiments of active pumping systems within a spacer may be illustrated in exemplary FIGS. 9A-E wherein the driving force to enable the active pumping system is provided through an externally coupled magnetic field.

For example, exemplary FIGS. 9A and 9B may illustrate an active, electromechanical pumping system, wherein the pumping motor utilizes an electromagnetic coupling to rotate the motor. Such coupling may be disposed within the fabric control band around the knee exterior (FIG. 5). In one exemplary embodiment a rotating magnet pump housed inside the implanted spacer can be implemented as either a ferro fluid (see Hatch, et al. “A Ferrofluidic Magnetic Micropump,” 10 J. of Microelectromechanical Systems (2000)) or a precisely fit set of cylinders that rotate with a pumping chamber for delivery through a 360-degree translation (see FIG. 9A). Alternatively, the pumping chamber may be driven by a rotating magnet, such as illustrated in exemplary FIG. 9B from S. H. Kim, et al, “Centrifugal Force Based Magnetic Micro-Pump Driven by Rotating Magnetic Fields,” 2011 J. Phys.: Conf. Ser. 266 012072. These represents examples of a remotely coupled magnetically driven micro pump that could be placed within an implanted spacer.

FIG. 9C may depict a peristaltic pump, whereby fluid flows through a tube compressed in sequence by several actuators. See, e.g., Meng et al, “Micro- and nano-fabricated implantable drug-delivery systems,” 3 Ther. Deliv. 1457-1467 (2012). These actuators could be subject to progressive magnetic forces to induce their movement thereby generating the pumping mechanism.

As illustrated in FIGS. 9D-9E, the prosthetic knee spacer may include a magnetically controlled delivery system that incorporates a compliant, elastomeric reservoir (CR), an elastomeric infusion cell (IC), and magnetically active pinch valve within the implanted spacer. This implant is externally controlled through a simple electromagnetic control system (CS) housed in an external knee band the patient must wear. The CR and IC may form drug compatible elastic balloons of specific characteristics acting in a two-stage balloon-like pump configuration.

The IC can be sized for administering a fixed liquid volume delivery; for example, 1.5 ml per period. In some exemplary embodiments, the delivery period may be set for a once a day occurrence. The IC may be a very thin, low durometer, ballooning silicone tube. The IC may be easy to expand, and may generate approximately 10-20 mmHg pressure (PIC) when full. Its fill volume may be defined by the IC chamber, e.g. the balloon expands to contact the inner walls of the IC chamber at 1.5 ml. The IC may exhaust into the synovial fluid through an exposed side port incorporating a simple flow restrictor (non-precision, less concern with fouling or changing flow rate over time) to meter antibiotic outflow to support (i) IC filling and (ii) dosage rate.

The CR can be a thicker, higher durometer ballooning silicone tube than the IC. The CR may generate approximately 40-100 mmHg pressure (PCR) across its working volume (never completely emptying) to always reliably fill the IC (PCR-min>PIC-max). It can be sized to hold greater than nine times (9×) the IC volume for an intra-refill period of approximately 9-10 days, suitable to coordinate weekly refill office appointments.

The CR inlet may attach to a septum exposed on the side of the prosthetic knee spacer in the effective knee space (under the skin) for percutaneous needle injection. The physician may locate the septum by palpating, ultrasound imaging, or visualizing its bright radiopaque indicator ring under office-based fluoroscopy. After needle access and with a common pressure monitoring syringe/infusion set, the physician may confirm that the CR pressure is approximately 40-50 mmHg, indicating near empty. Knowing the expected fill volume, the physician can administer antibiotic into the CR while continuously monitoring the pressure as it increases to approximately 90-100 mmHg, for example. Should the pressure not change or increase significantly above this level before delivering the entire volume, the physician may respond to these error conditions and should investigate, e.g. aspirate contents and attempt refilling, may use contrast/imaging to assess the device, etc. The physician should always concurrently monitor the patient's synovial fluid parameters and serum laboratory values (standard of care).

The CR and IC flow may be separated but connected by a pinch-valve tube. There may be a single, spring loaded, normally closed pinch valve that controls the antibiotic flow between the CR and the IC. The magnetic valve body can be manipulated through the external fabric band control system's controlling electromagnetic field. The valve may be opened for a brief period of approximately 10-15 seconds to allow the CR to completely fill the IC. The valve can be closed, and the IC may exhaust the antibiotic daily dose volume into the knee over several minutes. In 24 hours, the daily cycle may begin again.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments may be made by those skilled in the art without departing from the scope of the invention as defined by the following claims.

1-20. (canceled)

21. A prosthetic knee spacer comprising:
a lower surface, the lower surface having a locking component configured to interlock with a tibial tray of an artificial tibial component;
an upper surface, the upper surface having a pair of condyle support platforms with respective articular geometries configured to receive a pair of condylar surfaces of an artificial femoral component; and
a fluid delivery system within an internal cavity positioned between the lower and upper surface, wherein the fluid delivery system comprises a reservoir configured to contain fluid; and.
the fluid delivery system further comprises components for interaction with a separate external control system, said external control systems comprising a system to impose a magnetic field on the knee spacer to facilitate at least one of a pumping or valve mechanism to control the delivery of fluid into the knee space.

22. The prosthetic knee spacer according to claim 21, wherein the reservoir is fabricated from a flexible material capable of expanding and conforming to the hollow portion surrounding the internal cavity.

23. The prosthetic knee spacer according to claim 21, wherein the reservoir is segmented into compartments configured to isolate different fluids contained within the reservoir.

24. The prosthetic knee spacer according to claim 21, wherein the fluid delivery system further comprises a filling septum operatively connected to the reservoir to provide access to the reservoir.

25. The prosthetic knee spacer according to claim 24, wherein the filling septum is configured to be locatable through a patient's skin in which the prosthetic knee spacer is implanted by palpating, X-ray, ultrasound imaging, or fluoroscopy.

26. The prosthetic knee spacer according to claim 21, wherein the fluid delivery system further comprises an active or a passive fluid delivery means for dispensing a fluid from the reservoir.

27. The prosthetic knee spacer according to claim 21, wherein the external control system comprises a power supply, control circuit, and a locating device to assure alignment with the implanted spacer.

28. The prosthetic knee spacer according to claim 21, wherein the fluid delivery system is configured to interact with components and systems exterior to the prosthetic knee spacer to refill, communicate with, and control the fluid delivery system.

29. The prosthetic knee spacer according to claim 21, wherein the fluid delivery system is configured to operatively connect with a pressure monitoring gauge during filling.

30. The prosthetic knee spacer according to claim 21, wherein the upper surface and lower surface are fabricated from ultra-high molecular weight polyethylene.

31. The prosthetic knee spacer according to claim 21, further comprising a wireless communication system within the inner cavity, wherein the wireless communication system comprises sensors for detecting device failure and/or sensors for detecting joint infection.

32. The prosthetic knee spacer according to claim 21, further comprising an adjustment system within the inner cavity, where the adjustment system is configured for modifying the height of the spacer in situ without surgery.

33. A method of delivering a fluid to a knee prosthesis of a patient, comprising:
positioning the prosthetic knee spacer according to claim 21 between a tibial tray of an artificial tibial component and a pair of condylar surfaces of an artificial femoral component of the knee prosthesis of the patient such that the lower surface of the prosthetic knee spacer interlocks with the tibial tray and the pair of condyle support platforms on the upper surface of the prosthetic knee spacer receive the pair of condylar surfaces,
the prosthetic knee spacer comprising a fluid delivery system in the internal cavity, the fluid delivery system comprising a reservoir configured to contain fluid, a septum operatively connected to the reservoir, and fluid delivery element for dispensing a fluid from the reservoir to outside the prosthetic knee spacer;
adding fluid to the reservoir in the prosthetic knee spacer before said positioning and/or after said positioning by applying an external force to the septum, inserting a hollow tube into the septum, and inserting fluid into the septum via the hollow tube; and
delivering the fluid to surrounding synovial fluid, soft tissues, cartilage, bone and surfaces of the prosthetic knee by dispensing the fluid from the reservoir with the fluid delivery element.

34. The method according to claim 33, further comprising maintaining the dispensed fluid in the synovial fluid over an elongated therapy period of up to 6 months by refilling the reservoir and controlling delivery into the synovial fluid.

35. The method according to claim 33, wherein the fluid comprises at least one component selected from the group consisting of antimicrobial agents, hyaluronic acid, analgesics, anti-inflammatories, steroids, and anticoagulants or chemical surfactants to breakdown biofilm.

36. A total knee prosthesis for performing a total knee arthroplasty, comprising:
an artificial femoral component having a pair of condylar surfaces configured to be secured to the distal end of a femur;
an artificial tibial component having a tibial tray configured to be secured to the proximal end of a tibia; and
the prosthetic knee spacer according to claim 21 for positioning between the pair of condylar surfaces and the tibial tray so that the lower surface of the prosthetic knee spacer interlocks with the tibial tray and the pair of condyle support platforms on the upper surface of the prosthetic knee spacer receive the pair of condylar surfaces.

37. The total knee prosthesis according to claim 36, wherein the prosthetic knee spacer further comprises a fluid delivery system within the internal cavity, and the fluid delivery system comprises a reservoir configured to contain fluid, a septum operatively connected to the reservoir, and fluid delivery means for dispensing a fluid from the reservoir to outside the prosthetic knee space.

38. The total knee prosthesis according to claim 37, wherein prosthetic knee spacer further comprises a wireless communication system within the inner cavity, and the wireless communication system comprises sensors for detecting device failure and/or sensors for detecting joint infection.
